(19) United States
(12) Patent Application Publication
(10) Pub. No.: US 2026/0193159 A1
(43) Pub. Date: Jul. 9, 2026

(54) A PROCESS FOR THE SYNTHESIS OF STILBENE AND INTERMEDIATES THEREOF

(71) Applicant: FERTIS INDIA PVT LTD, Hyderabad, Telangana (IN)

(72) Inventors: Rahul Raju KANUMURU, Hyderabad, Telangana (IN); Shaji George KOCHUMALAYIL, Hyderabad, Telangana (IN); Ravikumar SURANENI, Hyderabad, Telangana (IN); Murali Mohanarao VANA, Hyderabad, Telangana (IN)

(73) Assignee: FERTIS INDIA PVT LTD, Hyderabad, Telangana (IN)

(21) Appl. No.: 19/129,781

(22) PCT Filed: Jul. 5, 2023
(86) PCT No.: PCT/IN2023/050655

§ 371 Date: May 14, 2025

(30) Foreign Application Priority Data
Nov. 14, 2022 (IN) 202241065105

(51) Int. Cl. C07C 43/315; C07C 37/50; C07C 41/26

(52) U.S. Cl. CPC C07C 43/315; C07C 37/50; C07C 41/26

(57) ABSTRACT

The present invention discloses a process for the preparation of Stilbene such as Pterostilbene and Resveratrol. More particularly, the present invention discloses synthesis of Stilbene employing a novel intermediate, 5-(4-(1-ethoxyethoxy) styryl)-benzene 1,3 diol and 1-(4-(1-ethoxyethoxy) styryl)-3,5-dimethoxy-benzene.

### FIELD OF INVENTION

The present invention relates to a process for the preparation of Stilbene such as Pterostilbene and Resveratrol. More particularly, the present invention relates to the synthesis of Stilbenes employing novel intermediates, viz., 5-(4-(1-ethoxyethoxy) styryl)benzene-1,3-diol and 1-(4-(1-ethoxyethoxy) styryl)-3,5-dimethoxy-benzene.

### BACKGROUND AND PRIOR ART

Both resveratrol and pterostilbene are monomeric Stilbene having a 6-2-6 carbon skeleton with two phenyl rings linked by a double-bonded ethylene bridge. Resveratrol has three hydroxyls (—OH) groups, while pterostilbene has two methoxy (—OCH₃) groups and one —OH group on aromatic rings which are linked by a double-bonded ethylene bridge. Stilbene exist as monomeric, dimeric, trimeric, oligomeric, and polymeric forms, or as glycosides. Stilbene possess biological activities, such as antioxidant, anti-diabetic, anti-obesity, cardioprotective and neuroprotective. Resveratrol and Pterostilbene represent two of the monomeric Stilbenes which are well-studied in the art.

JMC, 2002, 45 (12), 2534-2542 discloses a process for preparing Pterostilbene by: condensing 3,5-Dimethoxybenzyltriphenylphosphonium bromide with 4-(tert-butyldimethylsilyloxy) benzaldehyde in tetrahydrofuran to yield 4′-(tert-Butyl dimethylsilyloxy)-3,5-dimethoxy stilbene; and treating the 4′-(tert-Butyl dimethylsilyloxy)-3,5-dimethoxy stilbene with tetra butyl ammonium fluoride to afford Pterostilbene.

CN 1948274 discloses a process for preparing Pterostilbene by condensing 4-benzyloxybenzaldehyde with 3,5-dimethoxybenzyl phosphonate in the presence of sodium hydride to yield 3,5-dimethoxy-4-benzyloxystilbene; and debenzylating 3,5-dimethoxy-4-benzyloxystilbene in presence of aluminum chloride and N,N-dimethylaniline in dichloromethane to obtain Pterostilbene.

CN 1955153 discloses a process for preparing Pterostilbene by condensing 3,5-Dimethoxybenzyl phosphonic acid diethyl esters with hydroxy benzaldehyde methoxymethyl ether to afford the substituted 3,5-Dimethoxy-4-methoxy-methyloxystyrylbenzene; and treating the 3,5-Dimethoxy-4′-methoxymethyloxystyryl benzene dissolved in methanol with Pyridinium p-toluene sulfonate (PPTS) to give Pterostilbene.

Indian Journal of Chemistry, Section B: 2002, 41B (11), 2395-2398 discloses a process for preparing Resveratrol by treating tri-O-methyl (—OMe) or tri-O-benzyl (—OCH₂Ph) Resveratrol with BBr₃ in dichloromethane to yield Resveratrol.

U.S. Pat. No. 7,253,324 discloses a process for preparing Resveratrol by treating tri-O-methyl (OMe) or tri-O-benzyl (OCH₂Ph) Resveratrol with AICl/N, N-dimethyl aniline to obtain Resveratrol.

Subbaraju et al. U.S. Pat. No. 8,524,782 B2 discloses a process for the preparation of Stilbene by (i) condensing 3,5-dialkyloxybenzyl phosphonates with 4′-O-tetrahydropyranyl benzaldehyde to obtain 3,5-alkyl-4′-O-tetrahydropyranyl Stilbene and (ii) followed by deprotection to yield Stilbene. However, this process results in poor yields.

The limitations of the above methods, disclosed in the prior arts suffers from scalability, selective deprotection, the use of expensive reagents and low yields.

Therefore, there remains a need in the art to provide a process that involves cost-effective, industrially scalable, less expensive and readily available reagents.

Accordingly, it is an objective of the present invention to provide a cost-effective and industrially scalable process for the preparation of Stilbene with desired purity and yield.

### SUMMARY OF THE INVENTION

In line with the above objective, the present invention provides a scalable process for the preparation of substituted Stilbene involving novel intermediate compounds viz, viz., 5-(4-(1-ethoxyethoxy) styryl) benzene-1,3-diol (4a) and 1-(4-(1-ethoxyethoxy) styryl)-3,5-dimethoxy-benzene (4b).

Accordingly, in an aspect, the invention provides a process for the preparation of Stilbene comprising;
(i) condensing 4-(1-ethoxyethoxy)benzaldehyde with a compound of formula 3;

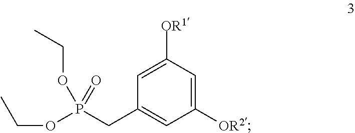

wherein, R¹ and R² are individually selected from the group consisting of hydrogen, methyl, aryl and arylalkyl, to obtain compound of formula 4; and

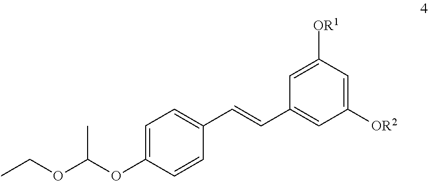

wherein, R¹ and R² are individually selected from the group consisting of hydrogen, methyl, alkyl, aryl and arylalkyl,
(ii) deprotecting the compound of formula 4 to yield Stilbene of formula 5

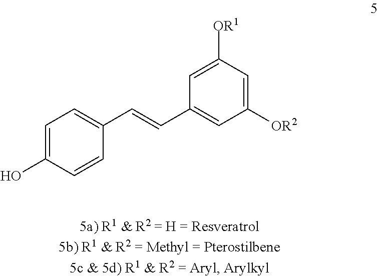

The process according to the present invention is depicted in Scheme below:

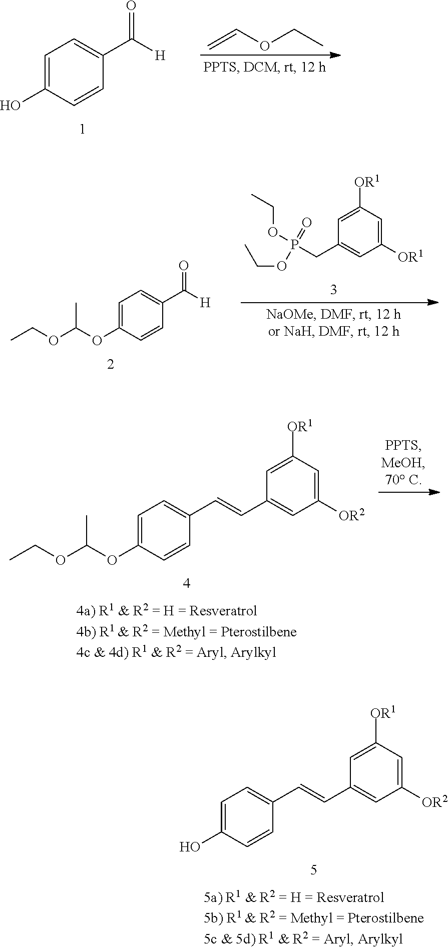

### DETAILED DESCRIPTION OF THE INVENTION

The invention will now be described in detail in connection with certain preferred and optional embodiments, so that various aspects thereof may be more fully understood and appreciated.

The present invention provides a scalable process for the preparation of substituted Stilbene involving novel intermediates viz, 5-(4-(1-ethoxyethoxy) styryl)benzene-1,3-diol (4a) and 1-(4-(1-ethoxyethoxy) styryl)-3,5-dimethoxy-benzene (4b).

Accordingly, in an aspect, the invention provides a process for the preparation of Stilbene comprising;
(i) condensing 4-(1-ethoxyethoxy)benzaldehyde with a compound of formula 3;

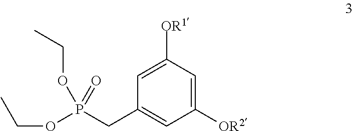

wherein, R¹ and R² are individually selected from the group consisting of hydrogen, methyl, alkyl, aryl and arylalkyl to obtain compound of formula 4; and

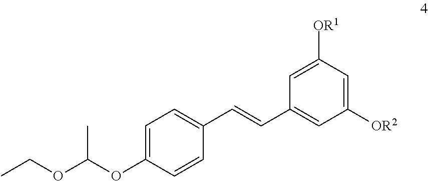

wherein, R¹ and R² are individually selected from the group consisting of hydrogen, methyl, alkyl, aryl and arylalkyl,
(ii) deprotecting the compound of formula 4 to yield Stilbene of formula 5

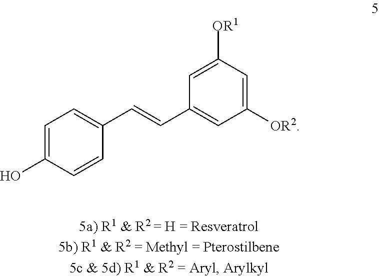

In an aspect, the compound of formula 3 (benzyl phosphonates) are selected from the group consisting of diethyl (3,5-dimethoxyphenyl) methyl phosphonate; diethyl (3,5 dihydroxyphenyl) methyl phosphonate; diethyl (3,5-diaryloxyphenyl) methyl phosphonate and diethyl (3,5-diarylalkoxyphenyl) methyl phosphonate.

In another aspect, the compound of formula (4) selected from the group consisting of;

a) 5-(4-(1-ethoxyethoxy) styryl)-benzene 1,3 diol (4a)

b) 1-(4-(1-ethoxyethoxy) styryl)-3,5-dimethoxybenzene (4b)

c) 1-(4-(1-ethoxyethoxy) styryl) 3,5-diaryloxybenzene (4c) and

d) 1-(4-(1-ethoxyethoxy) styryl)-3,5-diarylalkoxy-benzene (4d)

In yet another aspect, the compound of formula 5 is selected from pterostilbene (when R¹ and R²=methyl); and Resveratrol (when R¹ and R²=H).

The above intermediate compounds of formula (4), are the key intermediates for the synthesis of Stilbene such as Resveratrol and Pterostilbene.

In a further embodiment, the invention provides a process for the synthesis of compound of formula 4, which process comprises a step of condensing 3,5-dialkoxybenzyl phosphonates with 4′-O-ethylvinyl ether benzaldehyde in the presence of a base,

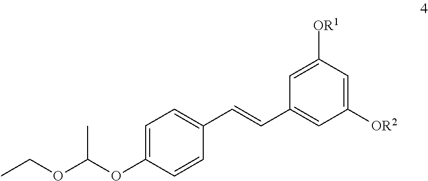

wherein R¹ and R² independently represent hydrogen, methyl, alkyl, aryl and arylalkyl.

In a further embodiment, the invention provides a novel intermediate compound, viz., 5-(4-(1-ethoxyethoxy) styryl)-benzene 1,3 diol (4a). This intermediate compound is prepared by a process which comprises a step of condensing diethyl (3,5 dihydroxyphenyl) methyl phosphonate with 4′-O-ethylvinyl ether benzaldehyde in the presence of a base,

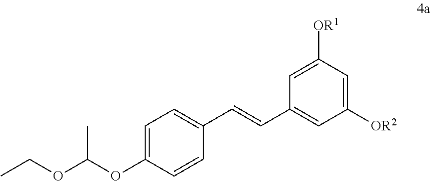

wherein R¹ and R² are hydrogen.

In yet another embodiment, the present invention provides a novel intermediate, viz., 1-(4-(1-ethoxyethoxy)-styryl)-3,5-dimethoxy-benzene (4b), which is characterized by spectral analysis by subjecting to ¹HNMR and Mass spectrometry which are described below.

¹HNMR (400 MHz, CDCl₃): δ 1.21 (3H, t, J=7.2 Hz), 1.51 (3H, d, J=5.2 Hz), 3.57-3.53 (1H, m), 3.81-3.70 (1H, m), 3.82 (6H, s), 5.42-5.38 (1H, m), 6.37 (1H, t, J=2.4 Hz), 6.65 (2H, d, J=2.0 Hz), 6.91 (1H, d, J=16.4 Hz), 7.00 (2H, d, J=3.2 Hz), 7.02 (1H, d, J=16.4 Hz), 7.43 (2H, d, J=8.8 Hz); Mass spectrum m/z: 329.2 (M+H)⁺.

In yet another embodiment, the invention provides a process for the preparation of 1-(4-(1-ethoxyethoxy)-styryl)-3,5-dimethoxy-benzene (4b), which process comprises; a step of condensing diethyl (3,5-dimethoxyphenyl) methyl phosphonate with 4′-O-ethylvinyl ether benzaldehyde in the presence of a base,

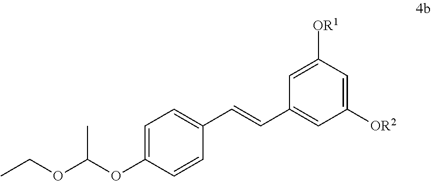

wherein R¹ and R² are methyl.

Accordingly, the process for the preparation of Stilbene includes preparation of 4-(1-ethoxyethoxy)benzaldehyde (2), which is prepared by the reaction of 4-hydroxybenzaldehyde and pyridinium p-toluene sulphonate in a suitable solvent with ethyl vinyl ether under stirring at room temperature to yield 4-(1-ethoxyethoxy)benzaldehyde. This compound was extracted into a suitable solvent(s) and isolated as an off-white solid in very good yield.

Further, preparation of a novel intermediate, 1-(4-(1-ethoxyethoxy) styryl)-3,5-dimethoxy-benzene (4b), involves reaction of 4-(1-ethoxyethoxy) benzaldehyde with diethyl (3,5-dimethoxyphenyl) methyl phosphonate in the presence of a base, such as NaOMe, K₂CO₃, LiHMDS, n-BuLi or NaH in a polar aprotic solvent such as DMF, DMSO, dioxane, etc., under stirring at room temperature for 12 h. After the completion of the reaction, the product was extracted into a suitable solvent such as ethyl acetate, washed with saturated brine solution and dried to afford 1-(4-(1-ethoxyethoxy) styryl)-3,5-dimethoxybenzene (4b), as colorless liquid. The product, 1-(4-(1-ethoxyethoxy) styryl)-3,5-dimethoxybenzene (4b), thus obtained was subsequently characterized by ¹HNMR and Mass spectrum.

Similarly, the preparation of another novel intermediate, viz., 5-(4-(1-ethoxyethoxy)-styryl)-benzene 1,3 diol (4a), involves reaction of 4-(1-ethoxyethoxy) benzaldehyde with diethyl (3,5-dihydroxyphenyl) methyl phosphonate in the presence of a base, such as NaOMe, K₂CO₃, LiHMDS, n-BuLi or NaH in a polar aprotic solvent such as DMF, DMSO, dioxane, etc., under stirring at room temperature for 12 h. After the completion of the reaction, the product was extracted into a suitable solvent such as ethyl acetate, washed with saturated brine solution and dried over sodium sulphate, to afford 5-(4-(1-ethoxyethoxy) styryl)-benzene 1,3 diol (4a), as a colorless liquid.

Further, preparation of 4-(3,5-dimethoxystyryl) phenol (5b, Pterostilbene), involves deprotection of 1-(4-(1-ethoxyethoxy) styryl)-3,5-dimethoxybenzene (4b) in an alcoholic solvent in presence of pyridinium p-toluene sulphonate under reflux for 2 h. The progress of reaction was monitored by TLC that indicates the deprotection of the ethyl vinyl ether group in compound 4b. Subsequently, the reaction mixture was cooled to room temperature; volatiles were evaporated and product was extracted into a suitable solvent such as ethyl acetate and purified to afford 4-(3,5-dimethoxystyryl) phenol (5b, Pterostilbene), as an off white solid.

In yet another embodiment, using the above process, Resveratrol was prepared, wherein, the solution of 5-(4-(1-ethoxyethoxy) styryl)-benzene 1,3 diol (4a) in methanol and pyridinium p-toluene sulphonate was refluxed for 2 h. The progress of reaction was monitored by TLC which shows the deprotection of the ethyl vinyl ether group from the compound 4a. The reaction mixture was cooled to room temperature, and the product was extracted into a suitable solvent such as ethyl acetate and purified to afford Resveratrol (5a), as an off white solid.

Thus, the process proposed by the present invention results in Stilbene such as Pterostilbene and Resveratrol in good yields. Moreover, the process proceeds through novel intermediates such as 5-(4-(1-ethoxyethoxy) styryl)-benzene 1,3 diol (4a) and 1-(4-(1-ethoxyethoxy) styryl)-3,5-dimethoxybenzene (4b)

The following example, which includes preferred embodiments, will serve to illustrate the practice of this invention, it being understood that the particulars shown are by way of example and for purpose of illustrative discussion of preferred embodiments of the invention.

### Example 1: Preparation of 4-(1-ethoxyethoxy) benzaldehyde (2)

To a stirred solution of 4-hydroxybenzaldehyde (10.0 g, 81.8 mmol) and pyridinium p-toluene sulphonate (2.06 g, 8.18 mmol) in dichloromethane (1.0 L), was added ethyl vinyl ether (17.71 g, 245.6 mmol). The reaction mixture was stirred at room temperature for 12 h. It was treated with water and extracted with dichloromethane (2 vol). Organic phase washed with saturated brine solution and dried over sodium sulphate dichloromethane was recovered under reduced pressure to afford 4-(1-ethoxyethoxy)benzaldehyde (2) as an off-white solid. Yield: 14.5 g (91%).

### Example 2: Preparation of 1-(4-(1-ethoxyethoxy) styryl)-3,5-dimethoxy-benzene (4b)

To a stirred solution of diethyl (3,5-dimethoxyphenyl) methyl phosphonate (13.34 g, 46.2 mmol) in DMF (30 mL) was added sodium methoxide (2.49 g, 46.2 mmol). The reaction mixture was stirred at 0° C. for 1 h and 4-(1-ethoxyethoxy)benzaldehyde (6.0 g, 30.8 mmol) in DMF solution was added drop wise at 0° C. The reaction mixture was stirred at room temperature for 12 h, added water and extracted in to Ethylacetatate (2 vol) and washed with saturated brine solution and dried over sodium sulphate. Ethylacetatate was recovered under reduced pressure to afford 1-(4-(1-ethoxyethoxy) styryl)-3,5-dimethoxybenzene (4b), as colorless liquid: 8.0 g (80%). ¹HNMR (400 MHz, CDCl₃): δ 1.21 (3H, t, J=7.2 Hz), 1.51 (3H, d, J=5.2 Hz), 3.57-3.53 (1H, m), 3.81-3.70 (1H, m), 3.82 (6H, s), 5.42-5.38 (1H, m), 6.37 (1H, t, J=2.4 Hz), 6.65 (2H, d, J=2.0 Hz), 6.91 (1H, d, J=16.4 Hz), 7.00 (2H, d, J=3.2 Hz), 7.02 (1H, d, J=16.4 Hz), 7.43 (2H, d, J=8.8 Hz), Mass spectrum m/z: 329.2 (M+H).

### Example 3: Preparation of 4-(3,5-dimethoxystyryl) phenol (Pterostilbene, 5b)

A stirred solution of 1-(4-(1-ethoxyethoxy) styryl)-3,5-dimethoxybenzene (4b) (3.2 g, 9.74 mmol) and pyridinium p-toluene sulphonate (0.49 g, 1.94 mmol) in MeOH (32 mL) was refluxed for 2 h. Progress of reaction was monitored by TLC showed the deprotection of the ethyl vinyl ether from the compound 3. Reaction mixture was cooled to rt, volatiles were evaporated and treated with water. It was extracted with ethyl acetate (2 vol), phases separated and organic phase washed sodium bicarbonate solution (2 vol), followed by 3 N aq. HCl (2 vol) and finally with saturated brine solution (50 mL). Organic layer was dried over Na₂SO₄ and evaporated under vacuum. The residue was purified to afford 4-(3,5-dimethoxystyryl) phenol (5b), as an off white solid. Yield: 1.9 g (66%). ¹HNMR (400 MHz, CDCl₃): δ 3.82 (6H, s), 4.99 (1H, bs), 6.38 (1H, t, J=2.4 Hz), 6.64 (2H, d, J=2.0 Hz), 6.82 (2H, d, J=8.8 Hz), 6.89 (1H, d, J=16.4 Hz), 7.02 (1H, d, J=16.4 Hz), 7.39 (2H, d, J=8.4 Hz), Mass spectrum m/z: 255 (M−H)⁻.

### Example 2: Preparation of 5-(4-(1-ethoxyethoxy)-styryl)-benzene 1,3 diol (4a)

To a stirred solution of diethyl (3,5-dihydroxyphenyl) methyl phosphonate (10.03 g, 38.5 mmol) in DMF (30 mL) was added sodium methoxide (2.08 g, 38.5 mmol). The reaction mixture was stirred at 0° C. for 1 h and 4-(1-ethoxyethoxy)benzaldehyde (5.0 g, 25.7 mmol) in DMF solution was added drop wise at 0° C. The reaction mixture was stirred at room temperature for 12 h, added water and extracted in to Ethylacetatate (2 vol) and washed with saturated brine solution and dried over sodium sulphate. Ethylacetatate was recovered under reduced pressure to afford 5-(4-(1-ethoxyethoxy) styryl)-benzene 1,3 diol (4a), as colorless liquid: 5.8 g (75%). ¹HNMR (400 MHz, CDCl₃): δ 1.23 (3H, t, J=7.2 Hz), 1.54 (3H, d, J=5.2 Hz), 3.47-3.43 (1H, m), 3.61-3.54 (1H, m), 5.01 (2H, bs), 5.42-5.38 (1H, m), 6.27 (1H, t, J=2.4 Hz), 6.55 (2H, d, J=2.0 Hz), 6.71 (1H, d, J=16.4 Hz), 7.05 (2H, d, J=3.2 Hz), 7.12 (1H, d, J=16.4 Hz), 7.33 (2H, d, J=8.8 Hz), Mass spectrum m/z: 301.3 (M+H)⁺.

### Example 4: Preparation of Resveratrol

A stirred solution of 5-(4-(1-ethoxyethoxy)-styryl)-benzene 1,3 diol (4a) (1.0 g, 3.33 mmol) and pyridinium p-toluene sulphonate (0.167 g, 0.66 mmol) in MeOH (10 mL) was refluxed for 2 h. Progress of reaction was monitored by TLC showed the deprotection of the ethyl vinyl ether from the compound 3a. Reaction mixture was cooled to rt, volatiles were evaporated and treated with water. It was extracted with ethyl acetate (2 vol), phases separated and organic phase washed sodium bicarbonate solution (2 vol), followed by 3 N aq. HCl (2 vol) and finally with saturated brine solution (50 mL). Organic layer was dried over Na₂SO₄ and evaporated under vacuum. The residue was purified to afford Resveratrol (4), as an off white solid. Yield: 0.55 g (65%). ¹HNMR (400 MHz, CDCl₃): δ 4.99 (1H, bs), 5.01 (2H, bs), 6.32 (1H, t, J=2.4 Hz), 6.68 (2H, d, J=2.0 Hz), 6.72 (2H, d, J=8.8 Hz), 6.81 (1H, d, J=16.4 Hz), 7.00 (1H, d, J=16.4 Hz), 7.31 (2H, d, J=8.4 Hz), Mass spectrum m/z: 227.2 (M−H)⁻.

We claim:

1. A process for the preparation of Stilbene comprising;
(iii) condensing 4-(1-ethoxyethoxy)benzaldehyde with a compound of formula 3 in presence of a base; 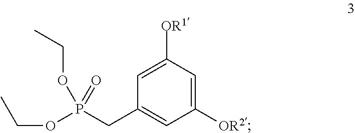
wherein, R¹ and R² are individually selected from the group consisting of hydrogen, methyl, alkyl, aryl and arylalkyl,
to obtain compound of formula 4; and 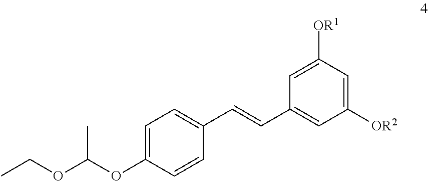
wherein, R¹ and R² are individually selected from the group consisting of hydrogen, methyl, alkyl, aryl and arylalkyl,
(iv) deprotecting the compound of formula 4 to yield Stilbene of formula 5

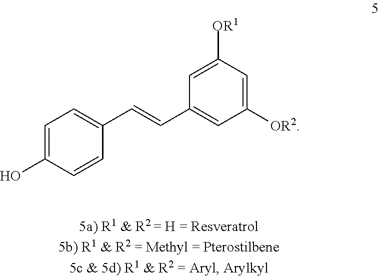

2. The process as claimed in claim 1, wherein, the compound of formula 3, (benzyl phosphonates) are selected from the group consisting of diethyl (3,5-dimethoxyphenyl) methyl phosphonate; diethyl (3,5 dihydroxyphenyl) methyl phosphonate; diethyl (3,5-diaryloxyphenyl) methyl phosphonate and diethyl (3,5-diarylalkoxyphenyl) methyl phosphonate.

3. The process as claimed in claim 1, wherein, the compound of formula (4) is selected from the group consisting of
a) 5-(4-(1-ethoxyethoxy) styryl)-benzene 1,3 diol (4a);
b) 1-(4-(1-ethoxyethoxy) styryl)-3,5-dimethoxy-benzene (4b);
c) 1-(4-(1-ethoxyethoxy) styryl) 3,5-diaryloxy-benzene (4c); and
d) 1-(4-(1-ethoxyethoxy) styryl)-3,5-diarylalkoxy-benzene (4d).

4. The process as claimed in claim 1, wherein, the base used in step a) is sodium methoxide.

5. The process as claimed in claim 1, wherein, the deprotection in step b) is conducted in an alcoholic solvent in the presence of pyridinium p-toluene sulphonate.

6. A compound of formula 4, 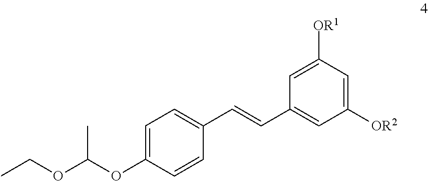
wherein R¹ and R² are hydrogen or methyl.

7. The compound of formula 4, as claimed in claim 6 is selected from the group consisting of
a) 5-(4-(1-ethoxyethoxy) styryl)-benzene 1,3 diol (4a);
b) 1-(4-(1-ethoxyethoxy) styryl)-3,5-dimethoxy benzene (4b).

8. A process for the synthesis of compound of formula 4, which process comprises a step of condensing benzyl phosphonate compound of 3 with 4′-O-ethylvinyl ether benzaldehyde (2) in the presence of a base, 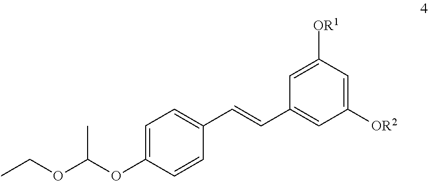
wherein R¹ and R² independently represent hydrogen or methyl.

9. The process as claimed in claim 8, wherein, the benzyl phosphonates are selected from the group consisting of 3,5-dimethoxybenzyl phosphonate (3b) and diethyl (3,5 dihydroxyphenyl) methyl phosphonate (3a).

10. The process as claimed in claim 8, wherein, the base is sodium methoxide.
